(19) United States
(12) Patent Application Publication
(10) Pub. No.: US 2007/0231301 A1
(43) Pub. Date: Oct. 4, 2007

(54) PARENTERAL LOW DOSE TYPE 1 INTERFERONS FOR BLADDER CANCER

(76) Inventor: Stephen L. Warren, Fort Collins, CO (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/692,624

(22) Filed: Mar. 28, 2007

Related U.S. Application Data
(60) Provisional application No. 60/788,130, filed on Mar. 31, 2006.

(51) Int. Cl. A61K 38/21; A61K 39/04

(52) U.S. Cl. 424/85.7; 424/248.1

(57) ABSTRACT

Novel methods and drug products for treating superficial bladder cancer (SBC) are disclosed, which involve parenteral administration of low doses of a type 1 interferon. The doses used are subtherapeutic for other solid tumors. Use of the novel methods and products in combination with other therapies for SBC is also described.

### REFERENCE TO CROSS RELATED APPLICATIONS

This application claims the benefit of priority under 35 USC § 119(e) of provisional patent application U.S.S.N.: 60/788,130 filed Mar. 31, 2006, the disclosure of which is hereby incorporated by reference in its entirety.

### FIELD OF THE INVENTION

This invention relates to the use of parenteral administration of Type 1 interferons for treating bladder cancer.

### BACKGROUND OF THE INVENTION

Bladder cancer is a significant public health problem, with a worldwide incidence of over 300,000 cases per year, and an incidence in the United States of more than 57,000 cases per year. In nearly 75% of patients with bladder cancer, the malignant cells are restricted to the inner surface of the bladder, and have not invaded into the muscle layer beneath the epithelium. Low stage bladder tumors meeting this description are referred to as “superficial bladder cancer” (“SBC”). Based upon microscopic features, most bladder tumors are classified as transitional cell carcinomas (TCCs), and the vast majority of grossly visible lesions have a papillary (cauliflower-like) morphology that is most readily appreciated when the cancer tissue is visualized under the microscope.

The mainstay of diagnosis and therapy for SBC involves cytoscopic identification and resection of visible tumors using a technique referred to as “transurethral resection of the bladder tumor” or “TURBT”. The TURBT technique works well to eliminate grossly visible papillary tumors, but it routinely fails to eradicate multifocal microscopic papillary lesions, and carcinoma in situ (CIS), two types of neoplastic lesions that may be completely invisible when the mucosa is examined with a cystoscope. Therefore, despite the use of TURBT, approximately 65% of SBC patients experience tumor recurrence within 5 years of diagnosis. Furthermore, the 3-year recurrence rate may exceed 80% for particularly aggressive tumor subtypes, which include Grade 3 lesions, tumors larger than 3 cm, previously recurrent tumors, multifocal tumors, or CIS. By comparison, solitary, low grade and stage, papillary SBC tumors recur within 18 months of TURBT in approximately 30% of cases.

Most SBC patients survive more than a decade after the initial diagnosis, and many have no evidence of tumor for periods of months to years between relapses. Thus, SBC may be viewed as a chronic disease, characterized by repeated treatments and relapses. The chronic nature of this disease means that its prevalence is about ten times its incidence, making SBC one of the most costly of all cancers.

There is a great need to reduce tumor recurrence following TURBT, because each time a tumor recurs, there is increased risk that the tumor will progress to muscle invasive or metastatic disease. Two common approaches to reduce the risk of recurrence are adjuvant intravescial chemotherapy or adjuvant intravesical immunomodulation.

In adjuvant intravesical chemotherapy, a cytotoxic agent, typically doxorubicin, valrubicin, thiotepa or mitomycin C, is introduced into the bladder via a catheter inserted into the urethra, and then washed out so that toxic exposure is limited primarily to the inner surface of the bladder. Cytotoxic agents reduce the recurrence rate by up to 30%, but none of these agents significantly reduces the rate of tumor progression.

Intravesical immunomodulation typically involves introducing an immunomodulator into the bladder within a few weeks of the TURBT procedure. The immunomodulator installation is maintained for about two hours before the patient voids.

The most commonly used immunomodulater is a specific strain of live, attenuated bovine tuberculosis (Mycobacteria bovis), usually referred to as “bacillus Calmette Guerin” (“BCG”). The mechanism of BCG action is not well understood, but probably involves stimulation of the adaptive and/or innate immune system, resulting in a localized T-cell, and possibly NK cell, -mediated destruction of cancer cells lining the inner surface of the bladder. In previously untreated patients with SBC, BCG adjuvant therapy reduces the rate of tumor recurrence to the extent that nearly 75% of patients remain disease free for the first two years. However, the effect of BCG is not durable, and at least half of all SBC patients receiving BCG will relapse within 5 years. Also, BCG therapy has an unfavorable adverse event profile. For example, approximately 90% of patients treated with BCG develop cystitis, an unpleasant and often painful irritation of the bladder mucosa, and about 5% of BCG-treated patients develop serious infectious complications, including BCG sepsis (i.e. disseminated tuberculosis), which is difficult to treat, and in rare cases, leads to death.

Interferon alpha (IFN-α) is a different type of immunomodulator that has been used to treat SBC. When doses of between 10-50 million international units (MIU) of IFN-α protein are introduced into the bladder via a catheter, IFN-α is relatively well tolerated, causing low grade fever and/or mild flu-like symptoms in some patients. Single agent intravesical interferon-alpha protein has modest efficacy in the first line setting (40% complete response rate), but tumor responses are rarely, if ever, durable. Also, this therapy is significantly less efficacious in patients who have failed BCG, with complete response rates of 15-20% and 12%, at one and two years, respectively.

Due to its modest efficacy, single agent intravesical IFN-α protein is rarely used to treat SBC; rather, intravesical instillation of IFN-α protein is more often used in combination with BCG to improve the therapeutic index of BCG. The treatment regimen for this combination therapy typically comprises an induction cycle of at least 6 weekly instillations of full strength BCG plus 5 to 50 MIU IFN-α protein, followed by several months to years of maintenance therapy with reduced-strength BCG (e.g., 1/10 to ⅓ strength) plus 5 to 50 MIU IFN-α protein. Various instillation schedules have been employed during maintenance therapy, ranging from 3-week cycles of weekly instillations begun at 3, 9 and 15 months after completion of the induction cycle (O'Donnell M A., et al., Interim results from a national multicenter Phase III trial of combination bacillus Calmette-Guein plus interferon-alpha 2B for superficial bladder cancer. J. Urol. 172: 888-893 [2004]) to monthly instillations at 9, 12, 18 and 24 months following initiation of therapy. (Mohanty N K., et al. Combined low-dose intravesical immunotherapy (BCG+interferon alpha-2b) in the management of superficial transitional cell carcinoma of the urinary bladder: a five-year follow-up. J. Chemotherapy 14: 194-197 [2002]).

In previously untreated SBC patients receiving BCG in combination with IFN-α protein, at least 70% will have no evidence of disease for a year after treatment, and more than half will have no evidence of disease 3 years after treatment. These results indicate that intravesical IFN-α potentiates the activity of BCG, but the mechanism is poorly understood. Type 1 interferons, including IFN-α, are known to prime the immune system by activating, directly or indirectly, a large number of immunologically relevant genes, including genes involved in Th1 type immune responses (TNF-alpha, IFN-gamma, IP-10). Consistent with an immune priming mechanism, it is believed that BCG and IFN-α protein must be administered concurrently to achieve a maximal therapeutic effect.

The ability of IFN-α to potentiate the biological activity of BCG has also been exploited in an attempt to reduce the toxicity of BCG. Lower doses of BCG have a better safety profile as compared to full dose BCG, including less frequent and less severe cystitis, and a lower risk of serious adverse events, such as BCG sepsis. Clinical studies have suggested that lower doses of BCG (e.g. ⅓^{d} or 1/10^{th} strength) combined with intravesical INF-alpha therapy may achieve an efficacy profile that is comparable to monotherapy with full dose BCG (Marinez-Pineiro, J. A et al, Long-term follow-up of a randomized prospective Trial comparing a standard 81 mg dose of intravesical bacille Calmette-Guerin with a reduced dose of 27 mg in superficial bladder cancer. 2002. BJU International, 89:671-680.); however, this has not been validated by large scale randomized clinical trials.

IFN-α, administered parenterally, has been used to treat other malignant diseases, including hairy cell leukemia, malignant melanoma, renal cell carcinoma, chronic myelogenous leukemia, essential thrombocythemia, polycythemia vera, non-Hodgkins lymphoma, carcinoid syndrome and AIDS related Kaposi's sarcoma. However, parenteral administration of IFN-α protein is not an accepted therapy for SBC, partly because the presumed target cells for this protein, the malignant urothelial cells, are located on the inner surface of the bladder, and partly because of the greater prevalence and severity of side effects associated with this route of administration at the doses approved for treating other solid tumors (O'Donnell, M A., Combined bacillus Calmette-Guerin and interferon use in superficial bladder cancer. Expert Rev. Ther. 3(6):809-821 [2003]). These side effects include acute flu-like symptoms, fever, myalgia (muscle aches), anorexia (loss of appetite), nausea, vomiting, myelosuppression (bone marrow toxicity, neutropenia, thrombocytopenia, anemia), liver toxicity (elevated liver enzymes) fatigue and psychological symptoms (lack of concentration, depression, anxiety). While the risk benefit ratio of high-dose systemic therapy with IFN-α is deemed acceptable for treating many types of solid tumors, it is not believed to be acceptable for treating SBC, partly because urologists, the specialists who treat SBC, are not accustomed to managing patients with the variety of side effects associated with the parenteral administration of high-doses of IFN-α.

None of the discussed above therapies for SBC are considered to be optimal due to low to modest efficacy or poor tolerability. Thus, a need exists for additional therapies for treating SBC.

### SUMMARY OF THE INVENTION

The present invention provides novel methods and medicaments for treating SBC, which employ parenteral administration of a type 1 interferon at doses that are lower than doses shown to be therapeutic for other solid tumors. The invention is based upon the rationale that the primary target cells of the interferon are not the malignant urothelial cells, but rather a host of immunocytes, including, but not limited to, T-lymphocytes, macrophages, professional antigen presenting cells, and natural killer cells. These immune cells traffic into and out of the bladder submucosa, regional (pelvic) lymph nodes and possibly the blood and lymphatic vascular systems. However, the inventor herein has deduced that intravesical administration of an interferon protein does not expose these immune cells to interferon for a sufficient time, or to a sufficient concentration of interferon, to provide optimal efficacy in treating SBC. In contrast, the parenteral route of administration is capable of providing interferon for a sufficient time, and at a sufficient concentration, to achieve the desired effects.

Thus, in one embodiment, the invention provides a method for treating a patient diagnosed with superficial bladder cancer (SBC), which comprises parenterally administering to the patient a type 1 interferon. The interferon is administered using a dosing regimen designed to provide an amount of the interferon that is within a pre-defined range for that interferon. The lower limit of the pre-defined range is the amount sufficient to achieve at least half maximal binding of the type 1 interferon receptor (IFNAR) on immune cells in the bloodstream for at least 1 day and the upper limit of the range is a dose of the type 1 interferon that is subtherapeutic for other solid tumors. In preferred embodiments, the methods of the invention include administration of chemotherapeutic agents or other immunomodulators such as BCG. Methods of the invention may be used alone, as adjuvant therapy following surgical resection of one or more tumors in the bladder, or as neoadjuvant therapy preceding surgery and/or BCG instillation.

In another embodiment, the invention provides a manufactured drug product for treating superficial bladder cancer (SBC). The drug product comprises (i) a pharmaceutical formulation comprising a type 1 interferon; and (ii) product information which comprises instructions for administering the pharmaceutical formulation to SBC patients according to a dosing regimen designed for that interferon. The dosing regimen is capable of providing an amount of the interferon that is within a pre-defined range for that interferon, wherein the lower limit of the range is the amount sufficient to achieve at least half maximal binding of the interferon α/β receptors on immune cells in the bloodstream for at least 1 day and the upper limit of the range is a dose that is subtherapeutic for other solid tumors. In some preferred embodiments, the drug product also comprises at least one other pharmaceutical formulation, which comprises a chemotherapeutic agent or a different immunomodulater.

A still further embodiment of the invention is a method of manufacturing a drug product for treating SBC, the method comprising: combining in a package a pharmaceutical formulation comprising a type 1 interferon and prescribing information. The prescribing information comprises instructions for parenterally administering the formulation to a patient diagnosed with SBC using a specific dosage regimen. The dosing regimen is capable of providing an amount of the interferon that is within a pre-defined range for that interferon, wherein the lower limit of the range is the amount sufficient to achieve at least half maximal binding of the interferon α/β receptors on immune cells in the bloodstream for at least 1 day and the upper limit of the range is a dose that is subtherapeutic for other solid tumors.

Another embodiment of the invention is the use of a type 1 interferon in the manufacture of a medicament for treating superficial bladder cancer. The medicament is formulated to parenterally deliver an amount of the type 1 interferon that is within a pre-defined range for the type 1 interferon, wherein the lower limit of the range is the amount sufficient to achieve at least half maximal binding of the interferon α/β receptors on immune cells in the bloodstream for at least 1 day and the upper limit of the range is a dose of the 1 interferon that is subtherapeutic for other solid tumors.

### DETAILED DESCRIPTION OF THE INVENTION

The present invention provides a novel approach to treating SBC with type 1 interferons, which employs parenteral administration of the interferon protein rather than intravesical administration. As used herein, parenteral administration means an intravenous, subcutaneous, or intramuscular injection.

This new approach is designed to overcome what the inventor has deduced is a major drawback of direct intravesical instillation of interferon α: poor access of this protein to the immune cells (e.g., T-cells, macrophages, antigen presenting cells, and natural killer cells), which are located in the submucosa beneath the bladder epithelium, the pelvic lymph nodes that drain the bladder, and in the blood and lymphatic vascular systems. Most patients receiving parenteral interferon α at doses as low as 3-5 MIU experience one or more of the following side effects: acute flu-like symptoms, fever, myalgia (muscle aches), anorexia (loss of appetite), nausea, vomiting, myelosuppression (bone marrow toxicity, neutropenia, thrombocytopenia, anemia), liver toxicity (elevated liver enzymes) fatigue and psychological symptoms (lack of concentration, depression, anxiety). If intravesically administered interferon α, at doses that are typically two-to 10-fold higher, were absorbed to a significant extent into the systemic circulation, it would consistently lead to a high incidence of these side effects. However, the fact that intravesical administration of 10-50 MIU interferon α is relatively well tolerated indicates that absorption and systemic exposure are minimal. Thus, it can be inferred that the transitional bladder epithelium, even if dysplastic, presents a substantial barrier to interferon α. This inference is consistent with the known physiological properties of the human bladder mucosa, which is a barrier epithelium. Due to the low systemic absorption of intravesically administered interferon α, and the fact that this protein is voided from the bladder within two hours of installation, the inventor herein has concluded that parenteral administration of an interferon protein should be a more efficient method by which to expose the immune cells to type 1 interferons.

The present invention employs lower doses of a type 1 interferon than the doses parenterally administered in the treatment of other solid tumors. It is generally believed that the efficacy of type 1 interferons in treating solid tumors such as melanoma and renal cell carcinoma is due to the ability of these proteins to (1) directly induce apoptosis in cancer cells and inhibit blood vessel formation, which direct effects require sustained exposure to relatively high levels of type 1 interferons and the accompanying high risk for side effects, as well as (b) the indirect activity of type 1 interferons in stimulating the body's immune system to eliminate the cancer cells.

In contrast, the objective of the present invention is to activate the IFNAR and one or more intracellular signal transduction pathways (e.g. JAK/STAT pathway, IRS 1/2/PI3K, p38, CrkL and/or vav) in a patient's normal (i.e., nonmalignant) immune cells for a time period sufficient to prime the patient's anti-neoplastic immune response. The inventor herein expects that this priming effect can be achieved by providing plasma levels of interferon that are sufficient to achieve at least half maximal binding of the interferon IFNAR on the immune cells in the patient's bloodstream. Such IFN plasma levels can be provided by doses of a type 1 interferon that are less than the lowest therapeutically effective dose established for that interferon in the treatment of other solid tumors, and even by doses that are less than the lowest dose of that interferon recommended for treating hepatitis C and other viruses.

Thus, in one embodiment, the invention provides methods and medicaments for treating a patient diagnosed with superficial bladder cancer using parenterally administration of a type 1 interferon. The amount of the type 1 interferon administered is within a range that has been pre-defined for that particular type 1 interferon. The lower limit of the range is the amount sufficient to achieve at least half maximal binding of the IFNAR on immune cells in the bloodstream for at least 1 day (preferably at least 2 days, more preferably at least 4 to 7 days) and the upper limit of the range is a subtherapeutic dose (i.e., less than the lowest therapeutically effective dose) of the type 1 interferon for other types of solid tumors, and preferably a dose of type 1 interferon that is subtherapeutic for hepatitis C.

As used herein, the term “type 1 interferon” means any interferon protein (abbreviated “IFN”) that is capable of binding to and activating the human IFNAR (also referred to as the IFN-α/β receptor complex), which comprises two transmembrane subunits, IFNAR1 and IFNAR2 (see Domanski, P., et al., The type-I interferon receptor. The long and short of it., Cytokine Growth Factor Rev. 7:143-151 [1996]; Brierley, M. M. et al., IFN-α/β receptor interactions to biologic outcomes: understanding the circuitry. J. Interferon Cytokine Res. 22:835-845 [2002] and Stark, G. R. et al. How Cells respond to Interferons. Ann. Rev. Biochem. 67:227-264 [1998]). Upon binding to a type 1 interferon, the IFNAR1 and IFNAR2 oligomerize and activate signal transduction via intracellular Janu-associated kinases, signal transducers and activators of transcription (JAK/STAT pathway) as well as other pathways in certain cell types (e.g. IRS 1/2/PI3K, p38, CrkL, and vav).

Type 1 interferons useful in practicing the present invention include, but are not limited to, all naturally-occurring subtypes of the type 1 interferons that are expressed in human cells: IFN-α, IFN-β, IFN-ω, and IFN-κ (see Chen, J. et. al., Diversity and Relatedness Among the Type 1 Interferons. J. of Interferon & Cytokine Res. 24:687-698 [2004]). Preferably, the type 1 interferon is a human IFN-α. Particularly preferred human IFN-α subtypes are α-2a (GenBank Accession Number NP_{—}000596) and α-2b (GenBank Accession Number AAP20099), which may be recombinantly produced as mature polypeptides as described in U.S. Pat. No. 6,610,830. Mature IFN-α-2α is marketed as Roferon® A by Hoffmann-LaRoche, Nutley, N. J. and mature IFN-α-2b is marketed as INTRON® A by Schering Corporation, Kenilworth, N.J. Another recombinant IFN-α that is suitable for use in the present invention is IFN-α-2c marketed as Berofor® by Boehringer Ingelheim GmbH, Germany. IFN-β subtypes which may be used in the present invention include IFN-β-1α, marketed as AVONEX by Biogen Idec and IFN-β-1b, marketed as Betaferon in Europe by Schering AG.

The term “type 1 interferon” also includes biologically active polypeptide fragments of type 1 interferons, as well as chimeric or mutant forms of type 1 interferons in which sequence modifications have been introduced, for example to enhance stability, without affecting their ability to activate the IFNAR, such as consensus interferons as described in U.S. Pat. Nos. 5,541,293, 4,897,471 and 4,695,629, and hybrid interferons containing combinations of different subtype sequences as described in U.S. Pat. Nos. 4,414,150, 4,456,748 and 4,678,751. A commercially available consensus interferon is marketed as Infergen® (interferon alfacon-1) by Valeant Pharmaceuticals, Costa Mesa, Calif.

Also included within the meaning of “type 1 interferon” are any of the foregoing molecules that have been covalently modified (referred to herein as a “modified interferon”) to enhance one or more of its pharmacokinetic or pharmacodynamic properties, such as conjugates between a type 1 interferon and a water soluble polymer and fusions between interferon and a non-interferon protein. A non-limiting list of polymers that may comprise interferon-polymer conjugates useful in practicing the present invention are polyalkylene oxide homopolymers such as polypropylene glycols, polyoxyethylenated polyols, copolymers thereof and block copolymers thereof, dextran polyvinylpyrrolidones, polyacrylamides, polyvinyl alcohols, and carbohydrate-based polymers. Examples of interferon-polymer conjugates are described in U.S. Patent Application Publication No. US 2004/0030101 A1, U.S. Pat. Nos. 6,113,906, 6,042,822, 5,951,974, 5,919,455, 5,738,846, 5,711,944, 5,643,575, 4,917,888 and 4,766,106. Particularly preferred interferon-polymer conjugates are pegylated interferons, which are conjugates between polyethylene glycol (PEG) and a type 1 interferon, as further defined below. A preferred interferon fusion protein is Albuferon®, a fusion between human serum albumin (HSA) and IFN-α, which was created by Human Genome Sciences, Rockville, Md.

As used herein, the term “pegylated interferon” means a covalent conjugate between at least one PEG moiety and at least one type 1 interferon molecule. In some embodiments, the PEG moiety consists of a linear PEG chain; while in other embodiments, the PEG moiety has a branched structure. Use of a branched PEG moiety allows attachment of two PEG molecules to the interferon molecule via a single linkage, with the resulting conjugate typically referred to as PEG2-IFN (US 2004/0030101 A1) or U-PEG-IFN (6,113,906) or branched-PEG-IFN.

Pegylated interferons may be prepared using a PEG composition having an average molecular weight ranging from about 200 to about 66,000 daltons, with preferred average molecular weights between 2,000 and 45,000 daltons. In describing specific pegylated interferons herein, the average molecular weight of the PEG polymer moiety is designated with a number shown as a subscript following PEG, i.e., PEGₙ. Two preferred PEG polymers are linear PEG_{12,000} and branched PEG_{40,000}.

The conjugation reaction may be performed with a wide variety of commercially available pegylation linkers, which use chemistries that target specific moieties on proteins, such as specific amino acid side chains and the N-terminal amine. One preferred linker chemistry employs N-hydroxysuccinimide (NHS)-PEG, which forms amide bonds with lysine side chain groups and the N-terminus of the interferon. This chemistry is used to make PEGASYS® (interferon alpha 2a, Hoffmann-LaRoche, Nutley, N.J.) (see US 2004/0030101 A1).

A particularly preferred pegylated interferon for use in the present invention is PEG-Intron® (pegylated interferon alpha-2b, Schering Corporation), which is manufactured using succinimydyl carbonate (SC)-PEG_{12,000}. This linker forms urethane bonds between PEG_{12,000} molecules and interferon molecules (see U.S. Pat. No. 5,951,974). Pegylation with SC-PEG_{12,000} typically produces a mixture of positional isomers of single, linear PEG molecules attached to single interferon molecules at different amino acid residues (See, e.g., Grace et al., Structural and biologic characterization of Pegylated Recombinatn IFN-α2b, J. Interferon and Cytokine Research 21:1103-1115 [2001]). When pegylation is performed at mildly acidic conditions as described in U.S. Pat. No. 5,951,974, pegylation at His 34 of IFNα-2b is favored (Wylie et al., Carboxylated Histidine Is a pH-Dependent Product of Pegylation with SC-PEG, Pharmaceutical Research 18 (9):1354-1360 [2001]).

The ability of any particular type 1 interferon, as defined above, to activate the IFNAR may be tested using techniques well-known in the art, such as measuring mRNA or protein levels for genes whose expression is known to be induced by activation of the IFNAR. For example, biomarkers of biologically active type 1 interferons include IP10 and other IFN-α inducible proteins, 2′5′ oligoadenylate and neopterin in the plasma, and interferon-gamma in the urine and plasma. Such biomarker expression can also be used as surrogate pharmacodynamic endpoints in determining a dosing regimen for a particular type 1 interferon to provide interferon plasma levels required for half-maximal binding to the IFNAR in the bloodstream.

The parenteral administration may be accomplished using a variety of drug delivery technologies such as pharmaceutically acceptable solutions and suspensions, sustained release and controlled release formulations or technology, and any mechanical device that releases type 1 interferons into the circulation. Pharmaceutically acceptable compositions of interferon typically include diluents of various buffers having a range of pH and ionic strength, carriers solubilizers and preservatives, and may be provided as injectable solutions or as lyophilized powders which are reconstituted in an appropriate diluent prior to injection. See, e.g., U.S. Pat. Nos. 5,766,582, 5,762,923, 5,935,566, and 6,180,096. Sustained release delivery technologies for protein pharmaceuticals typically employ formulating the protein of interest in a biodegradable hydrogel such as SABER™ technology (Durect, Cupertino, Calif.) or biodegradable polymer matrices such as PolyActive™ technology (OctoPlus, Leiden, The Netherlands) and Atrigel™ (QLTI, Vancouver, Canada). Examples of mechanical devices useful in the delivery of protein pharmaceuticals include pulsatile electronic syringe drivers (e.g., the Provider Model PA 3000, Pancretec Inc., San Diego, Calif.), portable syringe pumps (e.g., the Graesby Model MS 16A, Graesby Medical Ltd., Waterford, Herts England), constant infusion pumps (e.g., Disetronic Model Panomat C-5) and implantable osmotic pumps such as the DUROS® implant (Alza, Mountain View, Calif.).

Selection of a drug delivery technology for use with a particular type 1 interferon will depend to a large extent on the pharmacokinetic properties of that interferon. For example, for nonmodified interferons, which are rapidly cleared from the bloodstream, constant infusion with a mechanical device or use of a sustained release delivery technology is preferred. Conversely, a traditional bolus injection is the preferred means of administering a pegylated IFN-α with a prolonged serum half-life, such as the PEG-Intron and PEGASYS products.

The above described type 1 interferons bind to the IFNAR with different affinities. Therefore, the minimum concentration of a particular type 1 interferon that is sufficient to achieve half maximal binding to the IFNAR will depend upon the binding affinity of that particular type 1 interferon for IFNAR. The half maximal binding concentration (Kd values) of various interferon α subtypes fall between 10⁻¹¹ M and 10⁻⁹ M, depending upon the cell type used in the experiment (Rubinstein, M. and Orchansky, P. CRC Crit. Rev. Biochem. 21:249-275 [1986]; Aguet, M. and Mogensen, K. E. in Interferons, ed. Gresser, I. (Academic, New York), Vol. 5, pp. 1-22 [1983]). For example, in experiments with Daudi and CaKi cell lines, half maximal binding of interferon α subtypes occurs between 0.1 and 1.0 nM (Pfeffer, L. M. et al. Biological Properties of Recombinant alpha-interferons: 40^{th} Anniversary of the Discovery of Interferons. Cancer Research 58:2489-2499 [1998]). The binding affinities for other type 1 interferons have been published (Cutrone, E. C. and Langer, J. A. Identification of Critical Residues in Bovine IFNAR-1 responsible for interferon binding. J. Biol. Chem. 276:17140-17148 [2002]; and Subramaniam, P. et. al. Differential recognition of the type 1 interferon receptor by interferons T and α for the disparate cytotoxicities. Proc. Natl. Acad. Sci. 92:12270-12274 [1995]), including studies comparing the ability of various type 1 interferons to displace radiolabeled interferon α2 from interferon α/β receptors on Daudi cells (Cutrone, E. C. and Langer, J. A. Contributions of cloned type 1 interferon receptor subunits to differential ligand binding. FEBS LETTERS 404:197-202 [1997]). In the latter experiments, the following IC₅₀ values (concentration at which half maximal displacement occurs) were reported: IFN-α2 (1.3×10⁻¹⁰ M), IFN-α8 (3.4×10⁻¹⁰ M), IFN-α1 (2.3×10⁻⁹ M), IFN-β (3.2×10⁻¹⁰ M), IFN-α Con1 (1.4×10⁻¹⁰ M) and IFN-ω (0.6×10⁻¹⁰M). Taken together, published studies indicate that, while the binding affinities of individual type 1 interferons vary, the half maximal concentration at which they bind to the IFNAR is typically between 10⁻¹¹ and 10⁻⁹ M (Rubinstein, M. and Orchansky, P. CRC Crit. Rev. Biochem. 21:249-275 [1986]; Aguet, M. and Mogensen, K. E. in Interferons, ed. Gresser, I. (Academic, new York), Vol. 5, pp. 1-22 [1983]; Cutrone, E. C. and Langer, J. A. Contributions of cloned type 1 interferon receptor subunits to differential ligand binding. FEBS LETTERS 404:197-202 [1997]; Subramaniam, P. et. al. Differential recognition of the type 1 interferon receptor by interferons T and α for the disparate cytotoxicities. Proc. Natl. Acad. Sci. 92:12270-12274 [1995]). Furthermore, the skilled artisan can readily determine the binding affinity for any type 1 interferon of interest using techniques well known in the art (Price, N. and Stevens, L. Fundamentals of Enzymology: Cell and Molecular Biology of Catalytic Proteins, 3^{rd} Ed (ISBN-13: 978-0-19-850229-6; published 11 Nov. 1999).

Similarly, doses of a particular type 1 interferon that are subtherapeutic for non-SBC tumors may be determined by reference to the scientific literature. A subtherapeutic dose of a particular type 1 interferon is one that is lower than the lowest parenteral dose of that interferon that is recommended by a regulatory agency for treating any solid tumor other than SBC, or the lowest experimental dose published in a peer reviewed journal. If there is no published data on doses of a particular type 1 interferon that are efficacious in treating other solid tumors, a subtherapeutic dose of that interferon for use in the present invention may be estimated by comparing the specific activity and pharmacokinetic (PK) characteristics of the interferon of interest with the specific activity and PK characteristics of an interferon for which efficacy data is available, such as IFNα-2b. A comparison of the PK characteristics for two type 1 interferons involves comparing the area under-the-curve (AUC) values from the concentration versus time curves (PK) profiles determined for the two molecules. For example, this type of estimate has been used to compare non-pegylated and pegylated interferon a2b. Thus, INTRON A at a dose of 9 MIU/week (3 injections, each 3 MIU) provides a level of pharmacokinetic exposure comparable to PEG-Intron, administered at a dose of 0.3 μg/kg/week (Glue P et al. Pegylated interferon-alpha2b: pharmacokinetics, pharmacodynamics, safety, and preliminary efficacy data. Hepatitis C Intervention Therapy Group. Clin Pharmacol Ther. 68:556-567 [2001])

Table 1 below lists the recommended (and in some cases experimental) dosage regimens for using INTRON® A, PEG-Intron and PEGASYS to treat various disease indications.

TABLE 1
Recommended Dosage Regimens for INF-α in hepatitis &
oncology indications

Hepatitis B
INTRON A: 30-35 million international units (MIU) per week,
administered subcutaneously or intramuscularly, for up to 16 weeks.
Hepatitis C
INTRON A or ROFERON: 3 MIU, administered subcutaneously or
intramuscularly, three times per week for up to 24 months.
PEG-Intron monotherapy: 1 μg/kg/week, administered subcutaneously,
for one year.
PEGASYS monotherapy: 180 μg/week, administered subcutaneously, for
up to 48 weeks.
High risk melanoma (adjuvant to surgery)
INTRON A: 20 m² MIU, administered subcutaneously, 5 times per week
for 4 weeks (induction), and 10 m² MIU 3 times per week for 48 weeks
(maintenance).
PEG-Intron (experimental regimen): 6 μg/kg/week, administered
subcutaneously, once per week for 8 weeks (induction course), and
3 μg/kg/week for 252 weeks (maintenance course).
Hairy cell leukemia
INTRON A: 2 MIU/m², administered subcutaneously or intramuscularly,
daily for up to 6 months.
Chronic myelogenous leukemia
INTRON A: 4-5 MIU/m², administered subcutaneously, daily to
hematological remission.
PEG-Intron: 6 μg/kg/week administered subcutaneously for up to a year.
AIDS-related Kaposi's sarcoma
INTRON A: 30 MIU/m², administered subcutaneously or intramuscularly,
3 times per week for up to 16 weeks.
Renal cell carcinoma
INTRON A: 9-10 MIU, administered subcutaneously or intramuscularly,
3 times per week for 4 weeks.
PEG-Intron (experimental regimen): 4.5-6 μg/kg/week.

The present invention contemplates parenterally administering each type 1 interferon according to a dosing regimen that is designed to provide an amount of the interferon that is within the above-described pre-defined range for that interferon. As used herein the term “dosing regimen” means a combination of (a) number of injections, (b) frequency of injections and (c) the dose to be given at each injection. Since the dosing regimen need only provide a an amount of interferon that is sufficient to achieve at least half-maximal binding of the IFNAR for at least one day (preferably 2 to 7 days), each component of the dosing regimen will depend to a large extent on the binding affinity of the interferon for the IFNAR, as discussed above, as well as the pharmacokinetic properties of the type 1 interferon in the selected drug delivery technology. For example, for a nonmodified IFN-α to be delivered via subcutaneous injection, a dosage regimen designed to achieve half-maximal binding of the IFNAR for at least two days may comprise injecting substantially identical doses of the IFN-α on each of two subsequent days, and preferably comprises continuous infusion of an appropriate dose of the IFN-α for 48 hours. In contrast, the dosing regimen for a pegylated IFN-α would typically comprise a single injection. The selection of an optimal dosage regimen for a particular type 1 interferon will typically be within the discretion of the attending health care provider, and may include consideration of a variety of patient-specific factors such as the stage of the SBC at the time of treatment, the treatment history for the patient, age, weight, and gender.

A preferred dosage regimen for unmodified interferon-α2a or 2b (e.g., the INTRON A or ROFERON products) comprises or consists essentially of 0.01 MIU<3 MIU over a period of two days, For PEG₁₂₀₀₀ interferon alpha 2a or PEG₁₂₀₀₀ interferon alpha 2b, a preferred dosage regimen comprises or consists essentially of a single injection of 0.1 μg/kg≦4.0 μg/kg. Other preferred dosage regimens for PEG₁₂₀₀₀ interferon-α2b comprise or consist essentially of a single subcutaneous injection of a dose of 0.1 μg/kg<1.0 μg/kg, 0.1 μg/kg≦0.75 μg/kg, or 0.1 μg/kg≦0.5 μg/kg, or 0.1 μg/kg≦0.25 μg/kg, or 0.25 μg/kg≦0.5 μg/kg, or 0.5 μg/kg≦0.75 μg/kg. For branched PEG_{40,000} interferon-α2a preferred dosing regimens comprise or consist essentially of a single subcutaneous injection of a dose of 1.0<180 μg. or 1.0<135 μg. or 5.0<90 μg. or 25.0≦50 μg. As used herein, the term “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, which do not materially change the basic or novel properties of the specified dosage regimen, method, or composition.

In some embodiments, the dosing regimen is repeated at least once, and preferably multiple times at the discretion of the treating healthcare provider. For example, the healthcare provider may choose to administer the type 1 interferon therapy for a time period that has been shown in a clinical trial to reduce the rate of tumor recurrence or increase the median time to tumor recurrence. Alternately, the healthcare provider may choose to continue the type 1 interferon therapy for up to two years, or as long as the patient does not exhibit significant side effects. In addition, the invention contemplates repeating the dosage regimen following tumor recurrence.

In some embodiments, the patient diagnosed with SBC is treatment naïve, meaning that the patient has not been treated previously for SBC. In other embodiments, the patient has relapsed following previous treatment for SBC; nonlimiting examples of such previous treatment include tumor resection alone or tumor resection followed by intravesical installation BCG and/or interferon installation.

The novel type 1 interferon therapy described herein may be used in combination with other therapeutic approaches: e.g., in a neoadjuvant fashion one week to several weeks prior to intravesical instillation of an immunomodulater such as BCG; as adjuvant therapy with or without BCG following resection of visible tumors, e.g., by the TURBT technique; and in combination with one or more chemotherapeutic agents administered intravesically. Several substrains of Mycobacterium bovis BCG are commercially available and may be used in conjunction with the present invention, such as the Connaught substrain (BCG Vaccine multi-dose vial and ImmuCyst®, Aventis Pasteur) and the TICE substrain (OncoTICE®, Organon). Typical BCG doses administered are between 10⁶ to 10⁹ CFU. The invention also contemplates administration of BCG doses lower than the approved dosage ranges of these products. Another immunomodulator that is being investigated as an intravesical agent is Keyhole-limpet hemocyanin (KLH), a highly antigenic respiratory pigment of the mollusc Megathura cranulata. The chemotherapeutic agent may be any agent having anti-tumor or anti-neoplastic or cytotoxic activity. Examples of chemotherapeutic agents commonly used in intravesical therapy include anthracyclines (doxorubicin, epirubicin, valrubicin) the direct alkyoator thiotepa and the intracellularly activated mitomycin C. Another potential therapy for use in treating SBC includes Photofrin-Mediated Photodynamic Therapy (PDT), which involves intravenous administration of photosensitizers with subsequent in situ intravesical activation by use of whole bladder laser therapy (WB-PDT) with visible light (630 nm).

In a particularly preferred embodiment of the invention, an SBC patient receives a single subcutaneous injection of 0.1 μg/kg<1.0 μg/kg PEG_{12,000} interferon-α2a or 2b 0.5 hours to 24 hours before instillation of a half- or one-third dose of intravesical BCG.

All patents and publications mentioned herein are incorporated herein by reference for the purpose of describing and disclosing, for example, the compounds, cell lines, constructs, and methodologies that are described in such patents and publications which might be used in connection with the presently described invention. These patents and publications are named solely for this descriptive purpose, and the inventor explicitly reserve the right to antedate such references by virtue of prior invention.

While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed is:

1. A method of treating a patient diagnosed with superficial bladder cancer (SBC), which comprises parenterally administering to the patient a type 1 interferon using a dosing regimen that provides an amount of the type 1 interferon that is within a pre-defined range for the type 1 interferon, wherein the lower limit of the range is the amount sufficient to achieve at least half maximal binding of the interferon α/β receptors on immune cells in the bloodstream for at least 1 day and the upper limit of the range is a subtherapeutic dose of the type 1 interferon for other solid tumors.

2. The method of claim 1, wherein the type 1 interferon is an interferon alpha.

3. The method of claim 2, wherein the lower limit of the range is the amount sufficient to achieve at least half maximal binding of the interferon α/β receptors on immune cells in the bloodstream for at least 3 days and the interferon alpha is a pegylated interferon alpha.

4. The method of claim 3, wherein the pegylated interferon alpha is PEG_{12,000}-interferon alpha 2a or PEG_{12,000}-interferon alpha 2b and the dosing regimen comprises a single injection of a dose of 0.1 μg/kg≦4.0 μg/kg.

5. The method of claim 4, wherein the pegylated interferon alpha is PEG_{12,000}-interferon alpha 2b and the dosing regimen comprises a single subcutaneous injection of a dose of 0.1 μg/kg<1.0 μg/kg.

6. The method of claim 5, wherein the dose is 0.1 μg/kg≦0.5 μg/kg.

7. The method of claim 6, wherein the dose is 0.1 μg/kg≦0.25 μg/kg.

8. The method of claim 5, wherein the dose is 0.25 μg/kg≦0.5 μg/kg.

9. The method of claim 3, wherein the pegylated interferon alpha is U-PEG_{40,000}-interferon alpha 2a or U-PEG_{40,000}-interferon alpha 2b and the dosing regimen comprises a single subcutaneous injection of a dose of 1.0<180 μg.

10. The method of claim 9, wherein the pegylated interferon alpha is U-PEG_{40,000}-interferon alpha 2a and the dosing regimen comprises a single subcutaneous injection of a dose of 1.0<135 μg.

11. The method of claim 10, wherein the dose is 5.0≦90 μg.

12. The method of claim 10, wherein the dose is 25.0≦50 μg.

13. The method of claim 1, further comprising repeating the dosage regimen every week for a time period of 1 to 260 weeks.

14. The method of claim 13, wherein the time period is 2 to 130 weeks.

15. The method of claim 14, wherein the time period is 4 to 65 weeks.

16. The method of claim 1, further comprising administering to the patient a therapeutically effective dose of a different antitumor agent.

17. The method of claim 16, wherein the different antitumor agent is an immunomodulator administered to the bladder between 24 hours before and 24 hours after administration of the first dose of the type 1 interferon.

18. The method of claim 16, wherein the type 1 interferon is a pegylated interferon α and the antitumor agent is an immunomodulator administered to the bladder between 24 hours before and 168 hours after administration of the first dose of the type 1 interferon.

19. The method of claim 18, wherein pegylated interferon α is selected from the group consisting of PEG_{12,000}-interferon alpha 2a, PEG_{12,000}-interferon alpha 2b, U-PEG_{40,000}-interferon alpha 2a and U-PEG_{40,000}-interferon alpha 2b.

20. The method of any of claims 17-19, wherein the immunomodulator is bacille Calmette Guerin (BCG).

21. The method of claim 20, wherein the BCG is administered in a dose less than 1×10⁶ CFU.

22. The method of any of claims 17-19, wherein the immunomodulator is a bladder tumor vaccine.

23. The method of claim 16, wherein the different antitumor agent is a chemotherapeutic agent administered to the bladder between 24 hours before and 24 hours after administration of the first dose of the type 1 interferon.

24. The method of claim 16, wherein the type 1 interferon is a pegylated interferon α and the different antitumor agent is a chemotherapeutic agent administered to the bladder between 24 hours before and 168 hours after administration of the first dose of the type 1 interferon.

25. The method of claim 1, wherein the patient has had at least one bladder tumor surgically removed.

26. The method of any of claim 1, wherein the patient has carcinoma in situ of the bladder.

27. The method of claim 1, wherein the patient has superficial carcinoma of the bladder with high grade pathological characteristics.

28. The method of claim 1, wherein the patient is a treatment-naive patient.

29. The method of claim 1, wherein the patient is a treatment-experienced patient.

30. The method of claim 29, wherein the patient relapsed after prior treatment with intravesical interferon and intravesical BCG.

31. The method of claim 1, wherein the type 1 interferon is a sustained release formulation of an unmodified interferon alpha.

32. The method of claim 31, wherein the sustained release formulation comprises a biodegradable polymer matrix.

33. The method of claim 1, wherein the type 1 interferon is a fusion protein comprising human serum albumin fused to an interferon alpha.

34. The method of claim 1, wherein the lower limit of the range is the amount sufficient to achieve at least half maximal binding of the interferon α/β receptors on immune cells in the bloodstream for at least 2 days, the type 1 interferon comprises an unmodified interferon alpha and the dosing regimen comprises a subcutaneous injection of 0.005 million international units (MIU)<1.5 MIU once every twenty-four hours for a period of at least 48 hours.

35. The method of claim 1, wherein the lower limit of the range is the amount sufficient to achieve at least half maximal binding of the interferon α/β receptors on immune cells in the bloodstream for at least 7 days, the type 1 interferon comprises an unmodified interferon alpha and the dosing regimen comprises a subcutaneous injection of 0.005 million international units (MIU)<1.5 MIU once every twenty-four hours for a period of at least 7 days.

36. The method of claim 1, wherein the lower limit of the range is the amount sufficient to achieve at least half maximal binding of the interferon α/β receptors on immune cells in the bloodstream for at least 2 days the type 1 interferon is an unmodified interferon alpha and the dosing regimen comprises administering a total dose of 0.01 MIU<3.0 MIU by continuous subcutaneous infusion over a period of 48 hours.

37. A manufactured drug product for treating superficial bladder cancer (SBC), which comprises:
a pharmaceutical formulation comprising a type 1 interferon; and
product information which comprises instructions for administering the pharmaceutical formulation to SBC patients according to a dosing regimen that provides an amount of the type 1 interferon that is within a pre-defined range for the type 1 interferon, wherein the lower limit of the range is the amount sufficient to achieve at least half maximal binding of the interferon α/β receptors on immune cells in the bloodstream for at least 1 day and the upper limit of the range is a subtherapeutic dose of the type 1 interferon for other solid tumors.

38. The drug product of claim 37, wherein the type 1 interferon is an interferon alpha.

39. The drug product of claim 38, wherein the interferon alpha is a pegylated interferon alpha.

40. The drug product of claim 39, wherein the pegylated interferon alpha is PEG_{12,000}-interferon alpha 2a or PEG_{12,000}-interferon alpha 2b and the dosing regimen comprises a single injection of a dose of 0.1 μg/kg<1.0 μg/kg.

41. The drug product of claim 40, wherein the pharmaceutical formulation is a lyophilized powder and the drug product further comprises a solvent for reconstitution of the powder.

42. The drug product of claim 40, wherein the dosing regimen comprises a single injection of a dose of 0.1 μg/kg<0.75 μg/kg.

43. The drug product of claim 39, wherein the pegylated interferon alpha is U-PEG_{40,000} interferon alpha 2a or U-PEG_{40,000} interferon alpha 2b and the dosing regimen comprises a single subcutaneous injection of a dose of 1.0<180 μg.

44. The drug product of claim 43, wherein the pegylated interferon alpha is U-PEG_{40,000} interferon alpha 2a and the dosing regimen comprises a single subcutaneous injection of a dose of 1.0<135 μg.

45. The drug product of claim 37, further comprising a second pharmaceutical formulation which comprises a different anti-tumor agent, and the product information further comprises a dosage regimen for intravesical administration of the different anti-tumor agent.

46. The drug product of claim 45, wherein the different anti-tumor agent is a BCG substrain.

47. A method of treating a patient diagnosed with superficial bladder cancer (SBC), which comprises parenterally administering to the patient a pegylated interferon alpha between 4 and 12 hours prior to intravesical BCG installation, wherein the pegylated interferon alpha is administered using a dosing regimen that provides an amount of the pegylated interferon alpha that is within a pre-defined range for the pegylated interferon alpha, wherein the lower limit of the range is the amount sufficient to achieve at least half maximal binding of the interferon α/β receptors on immune cells in the bloodstream for at least 3 days and the upper limit of the range is a subtherapeutic dose of the pegylated interferon alpha for other solid tumors.

48. The method of claim 47, wherein the pegylated interferon alpha is PEG_{12,000}-interferon alpha 2a or PEG_{12,000}-interferon alpha 2b and the dosing regimen comprises a single subcutaneous injection of a dose of 0.1 μg/kg<1.0 μg/kg.

49. The method of claim 47, wherein the pegylated interferon alpha is U-PEG_{40,000}-interferon alpha 2a or U-PEG_{40,000}-interferon alpha 2b and the dosing regimen comprises a single subcutaneous injection of a dose of 1.0<180 μg.

50. A method of manufacturing a drug product for treating superficial bladder cancer (SBC), the method comprising:
combining in a package a pharmaceutical formulation comprising a type 1 interferon and
prescribing information, which comprises instructions for parenterally administering the formulation to a patient diagnosed with SBC using a specific dosage regimen,
wherein the dosing regimen is capable of providing an amount of the type 1 interferon that is within a pre-defined range for that interferon, wherein the lower limit of the range is the amount sufficient to achieve at least half maximal binding of the interferon α/β receptors on immune cells in the bloodstream for at least 1 day and the upper limit of the range is a dose that is subtherapeutic for other solid tumors.
